(19) United States
(12) Patent Application Publication
(10) Pub. No.: US 2017/0172745 A1
(43) Pub. Date: Jun. 22, 2017

(54) IMPLANT CONNECTION

(71) Applicant: WALDEMAR LINK GMBH & CO. KG, Hamburg (DE)

(72) Inventor: Helmut D. LINK, Hamburg (DE)

(73) Assignee: Waldemar Link GmbH & Co. KG, Hamburg (DE)

(21) Appl. No.: 15/129,883

(22) PCT Filed: Mar. 25, 2015
(86) PCT No.: PCT/EP2015/056391

§ 371 Date: Sep. 28, 2016

(30) Foreign Application Priority Data
Apr. 1, 2014 (DE) 10 2014 206 151.2

(51) Int. Cl. A61F 2/30; A61F 2/36

(52) U.S. Cl. CPC A61F 2/30767; A61F 2/3609; A61F 2002/30107; A61F 2002/30332; A61F 2002/3098; A61F 2310/00029; A61F 2310/00491; A61F 2310/00407; A61F 2002/365; A61F 2/3094

(57) ABSTRACT

The invention relates to an implant component having at least one connection section. The connection section is coated at least partially with a TiNb coating and said coating has a thickness of 1-20 μm, preferably 1-6 μm. The invention also relates to a modular endoprosthesis comprising an implant component of this type.

### TECHNICAL FIELD

The present invention relates to an implant component having a connecting portion as is used in modularized implants, and to such a modularized implant.

### PRIOR ART

Contemporary endoprostheses often have a modular structure, i.e. they are assembled of at least two separately selectable parts. Such a structure provides these prostheses with high adaptability to the individual requirements for the treatment of a patient.

The advantage of the modular structure is that despite the diverse adaptability of the endoprosthesis to the anatomy and biomechanical conditions of a patient, only a relatively small number of different modules must be kept available. These modules can, for example, take different shapes and/or materials into account. As a result, such a modular structure has in particular the advantage that implants for any possible permutation do not have to be kept in stock or manufactured individually.

A modularly structured endoprosthesis is assembled with connecting portions. Cone connections have proved to be particularly successful in this regard. In cone connections, one implant component has a female cone and the other a male cone. When fitting this connection together, the leverage resulting from the cone angle is utilized to apply a normal force on the cone surfaces so as to achieve a frictional connection between the two cones. Consequently, such realized implant components are clamped together.

For example, cone connections can be found in hip endprostheses to connect individual implant components. Simple modular hip endprostheses have here an implant connection between the prosthesis shaft and the femoral head.

After the introduction of such modular endoprostheses, however, the occurrence of crevice corrosion and friction corrosion was observed in the cone connection, which at worst can lead to a fatal failure of the implant. It is assumed that the corrosion is caused by micro-movements between the connecting surfaces of two implant components and tension occurring between them. This results in cracks and wear of the protective oxide layer on the surface of the implant material, leading to the above-mentioned corrosion phenomena.

This was countered by changes to the prosthesis design and more precise manufacturing processes. In addition to the form accuracy, the surface condition of the contact surfaces was improved.

The multiple modular endoprostheses subsequently placed on the market have additionally a connecting piece arranged between the prosthesis shaft and the femoral head. Consequently, there is an implant connection between the prosthesis shaft and the connecting piece as well as between the connecting piece and the femoral head. Prosthetic systems having such a connecting piece were developed to allow a better adjustment of the version/anteversion, length and offset of the prosthesis to the needs of the patient.

In these multiple modular prostheses, corrosion occurs primarily at the interface between the prosthesis shaft and the connecting piece. It was also found in modular prostheses that corrosion phenomena occur not only in metal-metal pairings, but also in metal-ceramic pairings, for example in hip endprostheses with a metal shaft and a ceramic head.

As a consequence of corrosion, the release of metal ions, metal oxides, metal organophosphates and small metal particles, which in turn lead to increased mechanical abrasion, is also observed. Pain, aseptic loosening and negative knock-on effects for the surrounding tissue may occur as a result. Allergic reactions, which may also necessitate a modification of the prosthesis, can also occur.

In view of this, it was an object of the invention to provide an implant component that allows a connection to a further implant component, thereby, however, counteracting the known aforementioned drawbacks for the patient in modular prostheses.

### SUMMARY OF THE INVENTION AND PREFERRED EMBODIMENTS

In order to solve the above object, an implant component according to claim 1 as well as a modular endoprosthesis according to claim 8 are provided by the present invention.

The implant component according to the invention has at least one connecting portion which is at least partially coated with a TiNb coating.

Surprisingly, the connecting portion coated in accordance with the invention not only prevents secondary allergic reactions, but at the same time ensures a reliable frictional locking when the thickness is selected appropriately, and results in better protection against corrosion.

Similar to titanium, the titanium-niobium coating has a high degree of biocompatibility. However, the mechanical properties of this metal alloy are better suited for the presently provided coating. In particular, the coating has a desired low degree of hardness so that a deadlock between a hard coupling component and the coated male cone is ensured by cooperation of the original surface structure of the cone connection.

As already stated above, typically an attempt is made to achieve a reliable frictional locking by a correspondingly machined surface of the implant material in the connecting portion. In other words, due to the mechanical properties of the coating, normal force occurs when the connecting portion comes into contact with a complementary connecting portion of another implant component, which causes sufficient clamping action by the thus resulting frictional force between the surfaces of the connecting portions.

Usually nitride coatings are used to improve the abrasion properties of implant components. Therefore, they are used, for example, for the running surfaces of endoprostheses, where however other wear conditions prevail than in the connecting area. This may be one reason that nitride coatings did not produce satisfactory results.

The coating on the connecting portion preferably has a thickness of 1 to 20, preferably 1 to 6 μm, since a stable, continuous and resilient connection of the implant component can be achieved in this range. Preferably, the coating is applied by PVD methods or implantation methods known to the skilled person.

In a particularly preferred embodiment, the coating has a layer thickness of 3 to 6 μm and, more preferably, 3 to 5 μm.

The aim is that the thickness of the layer is chosen to be high enough so as to achieve a coating which withstands mechanical loads. At the same time, the aim is not to choose the coating too thickly so that the structures of the cone surfaces are not obstructed/disturbed and thus do not preclude a stable frictional locking between the surfaces of the connecting area.

In a further preferred embodiment, the connecting portion is formed by a female cone and/or a male cone. The connecting portion is realized therewith in such a manner that it is connectable with a complementary cone of another, further implant component.

The other implant component can also be an implant component according to the invention having a coated connecting portion, however, it can also be any other implant component from the prior art. In both cases, the effect of preventing corrosion of the connecting portion is basically achieved independently of the other implant component.

In a particularly preferred embodiment of the implant component, the connecting portion is realized rotationally symmetrical.

This allows an alignment of the implant component about the rotational axis of the connecting portion and thus permits a better adaptation to the patient-specific anatomy and the biomechanical requirements.

However, it may also be the case that a relative movement of the implant component about its longitudinal axis is precisely not desired. In such a case, the connecting portion does not have a rotationally symmetrical connecting portion to prevent rotation of the implant component according to the invention relative to the component to which the implant component is to be connected.

In a preferred embodiment, a connecting portion according to the invention is used in an implant component which comprises a metal alloy.

This has advantages in particular with metal alloys, in which, as is known, severed or detached components lead to negative consequences for the affected patient.

Accordingly, in a particularly preferred embodiment, the implant component comprises a cobalt-chromium alloy.

The connecting portion according to the invention is preferably used in joint endoprostheses. This may be the prosthesis shaft, a connecting piece, a joint head and/or any component of such a joint endoprosthesis.

Furthermore, a modular endoprosthesis is provided by the present invention, having at least one implant component according to the invention with a coated connecting portion according to one of the aforementioned embodiments.

Such a modular endoprosthesis has the initially mentioned advantages of a modular structure, however avoids corrosion damage and the negative consequences thereof.

The invention also provides the use of a TiNb coating to prevent corrosion on a connecting portion of an implant component, the coating having a thickness of 1 to 20, preferably 1 to 6 μm.

It is understood that the coating can be applied not just in the connecting area, but also to the implant surface going beyond the connecting area or to the entire implant surface. Such a coating has the advantage that it can be manufactured in an easier and thus less expansive manner since, for example, the costs of masking portions of the implant, which are not to be coated, are not required.

1-9. (canceled)

10. Implant component having at least one connection portion, the connecting portion being coated at least partially with a TiNb coating, and the coating having a thickness of 1 to 20, preferably 1 to 6 μm.

11. Implant component according to claim 10, in which the coating has a thickness of 3 to 6 μm, preferably 3 to 5 μm.

12. Implant component according to claim 10, in which the connecting portion is formed by a female and/or male cone.

13. Implant component according to claim 10, in which the connecting portion is rotationally symmetrical.

14. Implant component according to claim 12, in which the connecting portion is rotationally symmetrical.

15. Implant component according to claim 10, wherein the implant component has a metal alloy.

16. Implant component according to claim 12, wherein the implant component has a metal alloy.

17. Implant component according to claim 14, wherein the implant component has a metal alloy.

18. Implant component according to claim 10, wherein the implant component has a CoCr alloy.

19. Implant component according to claim 12, wherein the implant component has a CoCr alloy.

20. Implant component according to claim 13, wherein the implant component has a CoCr alloy.

21. Implant component according to claim 14, wherein the implant component has a CoCr alloy.

22. Implant component according to claim 10, wherein the implant component is a prosthesis shaft, a connecting piece or a joint head.

23. Implant component according to claim 14, wherein the implant component is a prosthesis shaft, a connecting piece or a joint head.

24. Implant component according to claim 17, wherein the implant component is a prosthesis shaft, a connecting piece or a joint head.

25. Implant component according to claim 21, wherein the implant component is a prosthesis shaft, a connecting piece or a joint head.

26. Modular endoprosthesis with at least one implant component according to claim 10.

27. Modular endoprosthesis with at least one implant component according to claim 14.

28. Modular endoprosthesis with at least one implant component according to claim 17.

29. Use of a TiNB coating to prevent corrosion on a connecting area of an implant component, wherein the coating has a thickness of 1 to 20, preferably 1 to 6 μm.
